(19) United States
(12) Patent Application Publication
(10) Pub. No.: US 2025/0033851 A1
(43) Pub. Date: Jan. 30, 2025

(54) NEW SINGLE-USE PACKAGING

(71) Applicant: NOTPLA LIMITED, London (GB)

(72) Inventors: Pierre-Yves PASLIER, London (GB); Rodrigo Garcia GONZALEZ, London (GB)

(21) Appl. No.: 18/709,172

(22) PCT Filed: Nov. 11, 2022
(86) PCT No.: PCT/GB2022/052872

§ 371 Date: May 10, 2024

(30) Foreign Application Priority Data
Nov. 12, 2021 (GB) 2116335.7

(51) Int. Cl. B65D 65/46; A61K 8/11; B65D 85/72

(52) U.S. Cl. CPC B65D 65/466; A61K 8/11; B65D 85/72; A61K 2800/87

(57) ABSTRACT

The invention relates to a liquid encapsulated within a membrane, wherein the liquid has a water content of less than 20 wt %, wherein the membrane comprises an extract derivable from seaweed or a cellulose polymer. The invention also relates to methods of preparing the same and uses thereof.

### FIELD OF INVENTION

The present invention relates to liquids encapsulated in a membrane comprising a extract derivable from seaweed or a cellulose polymer, and methods of producing the same, for single-use packaging consumer products, such as edible and cosmetic goods.

### BACKGROUND

Plastic packaging is used in abundance globally and large quantities of plastic end up being disposed of in landfill or incinerated, thus contributing to environmental damage such as marine pollution. The impact of plastic packaging on the environment is huge. It has been estimated that the societal cost of plastic produced just in 2019 to be $3.7 trillion (WWF, 2021) and that €13 billion a year of damage to global marine ecosystems is caused by plastic. Further, 80-85% of marine litter found on Europe's beaches is plastic (EU Single Use Plastics Directive, ISBN 978-92-76-12903-5).

Currently, a major cause of plastic not being recycled is the difficulty in recycling particular types of plastic, such as the thin plastic films used in packaging food products or other consumer products. There is therefore a need to develop environmentally friendly alternatives to plastic packaging.

Seaweed and cellulose are cheap and abundantly available materials that biodegrade very quickly and are thus excellent alternative materials to single-use and non-biodegradable or non-recyclable plastics.

### SUMMARY OF THE INVENTION

The present invention seeks to address the problem outlined above by developing membranes comprising seaweed extracts or cellulose polymers to encapsulate liquids. This results in a cost effective, environmentally sustainable solution for packaging and storing liquid consumer products, in particular as an alternative to plastic packaging such as sachets, bottles or tubes. The inventors have surprisingly found that liquids with particular properties (particularly those that have a water content lower than 20 wt %) can be encapsulated in a membrane comprising seaweed extracts or cellulose polymers and remain stably stored for significant periods of time without the liquid permeating out of the membrane. This allows the biodegradable membrane to replace plastic packaging of such liquid consumer products.

Accordingly, a first aspect of the invention is a liquid encapsulated within a membrane, wherein the liquid has a water content of less than 20 wt %, wherein the membrane comprises an extract derivable from seaweed.

A second aspect of the invention is a method of preparing a liquid encapsulated within a membrane, said method comprising the steps of:
(i). providing an extract derivable from seaweed solution;
(ii). pumping the solution of step (i) through a die to create two ribbons or films of gel;
(iii). injecting a liquid having a water content of less than 20 wt % between the two ribbons;
(iv). sealing the two ribbons around the liquid thereby forming the liquid encapsulated within a membrane.

A third aspect of the invention is the use of the liquid encapsulated in a membrane as provided in the first aspect or the container of the fourth aspect to:
(i). package and/or store the liquid; and/or
(ii). transport the liquid; and/or
(iii). provide a single dose of the liquid to a user.

A fourth aspect of the invention is a container comprising at least one liquid encapsulated within a membrane according to the first aspect of the invention.

### DESCRIPTION OF THE FIGURES

FIGS. 1A to 1F illustrate spherical shapes of a liquid encapsulated within a membrane comprising an extract derivable from seaweed.

FIGS. 2A to 2F illustrate ellipsoid shapes of a liquid encapsulated within a membrane comprising an extract derivable from seaweed.

FIGS. 3A to 3F illustrate oblong shapes of a liquid encapsulated within a membrane comprising an extract derivable from seaweed.

FIGS. 4A to 4F illustrate pipette shapes of a liquid encapsulated within a membrane comprising an extract derivable from seaweed.

FIGS. 5A to 5C illustrate sachet or pillow shapes of a liquid encapsulated within a membrane comprising an extract derivable from seaweed.

FIGS. 6A to 6C illustrate stand up (A), tetrahedral (B), or stick (C) pouch shapes of a of a liquid encapsulated within a membrane comprising an extract derivable from seaweed.

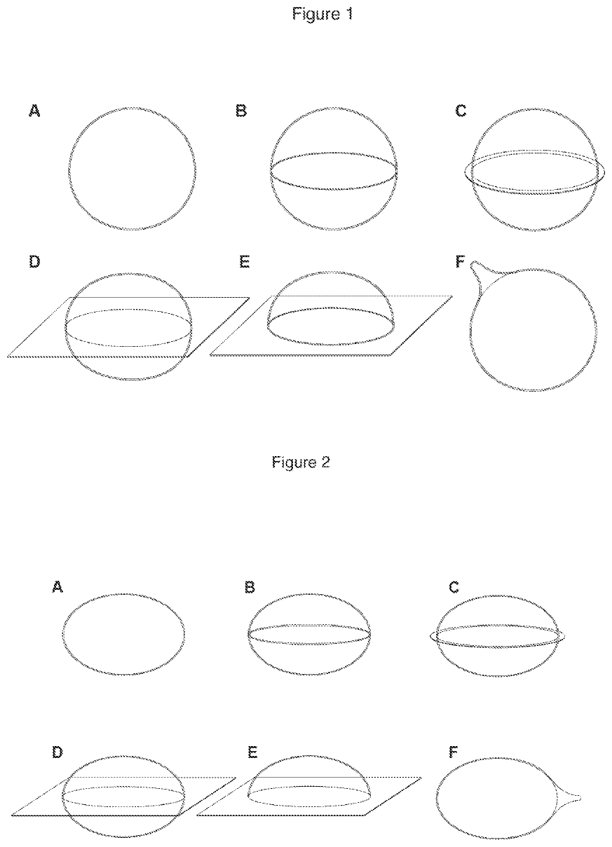

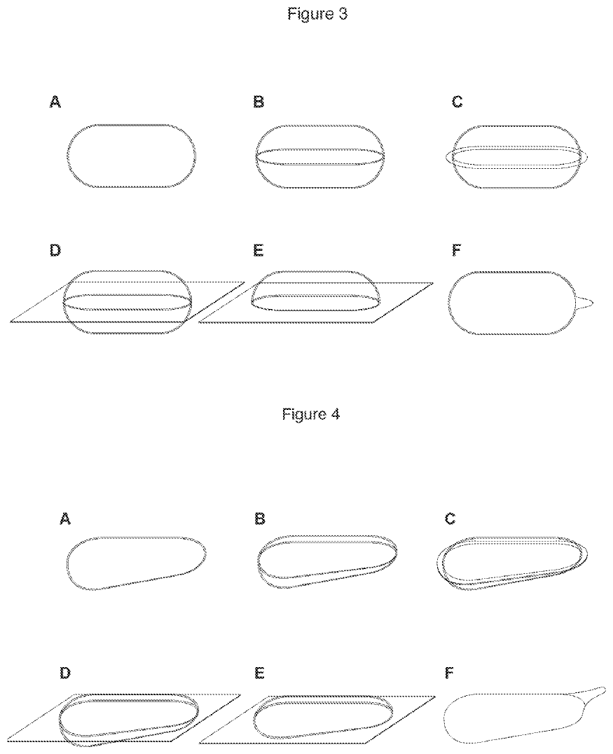

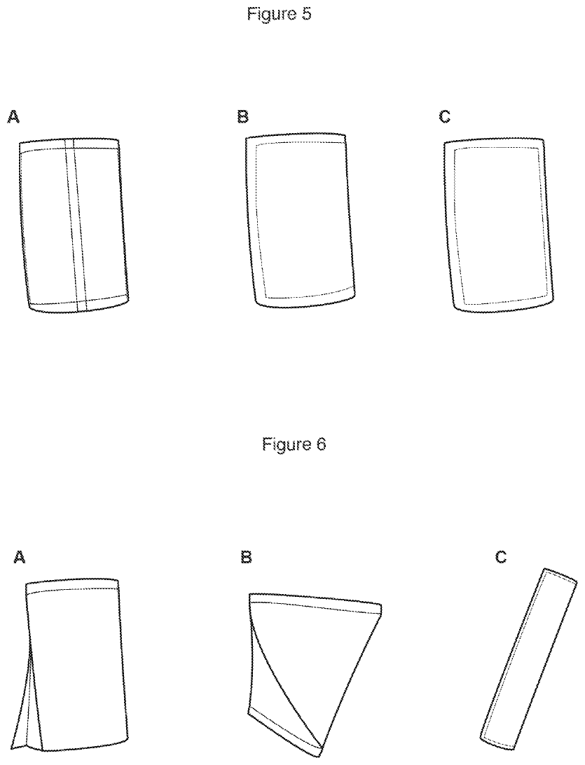

### DETAILED DESCRIPTION OF THE INVENTION

Unless indicated otherwise, all technical and scientific terms used herein will have their common meaning as understood by one of ordinary skill in the art to which this invention pertains.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof is to be understood to imply the inclusion of a stated element, integer or step, or group of elements, integers or steps, and the exclusion of any other element, integer or step or group of elements, integers or steps.

As used herein, the term “liquid” refers to a flowable product that may have a viscosity of up to 250,000 cps. As such, the term “liquid” is intended to encompass both low viscosity products such as oils (e.g. olive oil) and higher viscosity products such as pastes, slurries, suspensions, creams and sauces. The inventors have found that that the liquid may contain at most 20 wt % water. Liquids with more than 20 wt % water content have been found to dissolve the membrane.

Viscosity describes a liquid's thickness, more specifically its resistance to flow and is reported in centipoise (cP). Viscosity ranges for liquid categories are: thin (1-50 cP), nectar-like (51-350 cP), honey-like (351-1,750 cP), and spoon-thick (greater than 1,750 cP). Because the thickness of most consumable food based liquids changes as a function of its flow rate, viscosity measures are typically reported at a specific shear rate. These liquids are described as “shear-thinning” in that they decrease in viscosity as their flow rate increases.

“Viscosity” is typically measured at room temperature (e.g. 20° C.). The viscosity of certain liquids may be dependent on shear rate (e.g. shear thinning fluids). The term “liquid” as used herein is intended to cover shear rate-independent fluids which have a viscosity of up to 250,000 cps as well as shear thinning fluids which have a viscosity of up to 250,000 cps at a high shear rate (e.g. a shear rate of 10 s⁻¹ or 100 s⁻¹). Where viscosities of liquid products are discussed herein, the values mentioned are to be interpreted as viscosity at a high shear rate (e.g. a shear rate of 10 s⁻¹ or 100 s⁻¹) when applied to shear thinning fluids. The values herein are measured by dynamic (absolute) viscosity measurements. Dynamic viscosity is also known as absolute viscosity and most often relates to non-Newtonian fluids. It refers to the fluid's internal resistance to flow when force is applied. The dynamic viscosity can be measured using apparatus such as the following:
Falling piston viscometers: Falling piston viscometers use the force created by a falling piston to measure viscosity. They measure dynamic viscosity, because stress is applied to the fluid.
Rotational viscometers: A rotational viscometer measures how much torque is required to turn a spindle immersed in a fluid. The spindle applies stress to the fluid, resulting in a measurement of dynamic viscosity.
Falling ball viscometers: A falling ball viscometer measures the force required for a ball to fall through a fluid. The ball applies stress to the fluid, giving a measurement of dynamic viscosity. Patented in 1932 by Fritz Höppler, the falling ball viscometer was actually the first type of viscometer to measure dynamic viscosity.
Vibrational viscometers: Vibrational viscometers measure the resistance of a fluid to vibration. Since the vibration constitutes a force being applied to the fluid, these viscometers measure dynamic viscosity (Johnson, J. F. et al, 1975, Determination of Viscosity of Food Systems. Theory, Determination and Control of Physical Properties of Food Materials, 25-38).

The term “encapsulated” means being enclosed or surrounded by (e.g. in a membrane).

The term “membrane” means a thin layer, film or skin forming a barrier or lining. The membrane of the present invention comprises an extract derivable from seaweed. The encapsulated liquids according to the invention are also referred to as “film pods” or “soft gel capsules” herein.

“wt %” is a common abbreviation in the art to mean the “weight %” with respect to the total weight of the article/material referred to.

The present invention provides a liquid encapsulated within a membrane, wherein the liquid has a water content of less than 20 wt %, and wherein the membrane comprises an extract derivable from seaweed.

Suitably, the extract derivable from seaweed is sourced from one Gelidium, Gracilaria, Chondrus crispis, Eucheuma Denticulatum (Spinosum), Kappaphycus Alvarezii (Cottonii), Gigartina skottsbergii, Eucheuma cottonii, Gigartina stellata, Chondrus, Ulva, cladophora, codium, monostromea, prasiola, spongomorpha, preferably from Gracilaria, Gelidium, or Eucheuma Denticulatum, most preferably Gelidium or Euchema Denticulatum (Spinosum).

In preferred embodiments, the extract derivable from seaweed comprises, such as consist of, carrageenan, agar, or a cellulose polymer, such as hemicellulose, microfibrilated cellulose, microcrystalline cellulose, Hydroxypropylmethyl Cellulose (HPMC) or Carboxymethyl cellulose (CMC), preferably HPMC or CMC, preferably HPMC.

Hemicellulose is a heterogeneous polymer composed of many sugars, such as xylose, arabinose, mannose, and galactose, that are C5 and C6 sugars. Hemicellulose is known as the second most abundant carbohydrate material and consists of 25%-35% dry weight wood material. Hemicellulose may be derived from waste materials and also through seaweed extraction processes. In some embodiments, the extract derivable from seaweed comprises hemicellulose.

Carrageenan and agar are typically solely derived from seaweed by well-known techniques in the art. Cellulose polymers, such as microfibrilated cellulose, microcrystalline cellulose, Hydroxypropylmethyl Cellulose (HPMC) or Carboxymethyl cellulose (CMC), can be derived from cellulose extracted from seaweed, but can also be derived from other natural sources such as wood pulp or cotton, or it can be made artificially. All of these sources are suitable for use in the present invention. However, in a preferred aspect, the cellulose polymer is sourced from seaweed. HPMC, also known as hypromellose, is produced from cellulose, a natural polymer and fiber, which is considered to be safe for human consumption. HPMC is a water soluble nonionic cellulosic polymer in which some of the hydroxyl groups are substituted with methoxy and hydroxypropyl groups.

Cellulose is present in the cell walls of all seaweeds as the fibrillar skeleton material, including in green, red, and brown seaweeds. Cellulose is often present along with other diverse polysaccharides such as xylan, mannan, galactan, alginic acid, agar, carrageenan, rhamnose-glucuronate/iduronic; depending on the type of the seaweed group. The cellulose may be extracted separately or in conjunction with other useful polysaccharides such as starch, as described herein. For example, chlorophyceae members are known to have a wide array of cell walls including cellulose-pectin complexes and other cell walls made of hydroxyproline-rich glycoproteins which may offer specific benefits when extracts are used in combination. As a result, seaweed contains a wide array of ingredients, specifically cellulose, starch, agar and carrageenan that are valuable sources for creating biodegradable, natural materials.

In one embodiment, the extract derivable from seaweed does not comprise alginate, i.e. the membrane does not comprise alginate. Alginate is a well-known water-soluble biopolymer that is extracted from brown seaweed. In an alternative embodiment, the extract derivable from seaweed further comprises alginate, preferably in a low concentration such as from 0.01 wt % to 10 wt %. The addition of alginate to the membrane helps reduce cracking in the membrane and improve elasticity.

Suitably, the liquid may be water-based, glycerine-based or oil-based, preferably glycerine-based or oil-based. The terms “water-based”, “glycerine-based” and “oil-based” refer to a liquid that comprises water, glycerine, or oil, respectively.

In some embodiments, the liquid has a water content of less than 19 wt %, such as less than 18 wt %, 17 wt %, 16 wt %, 15 wt %, 14 wt %, 13 wt %, 12 wt %, 11 wt %, 10 wt %, 9 wt %, 8 wt %, 7 wt %, 6 wt %, 5 wt %, 4 wt %, 3 wt %, 2 wt % or 1 wt %, preferably less than 15 wt %, more preferably less than 13 wt %, more preferably less than 10 wt % water content.

In some embodiments, the liquid has a water content greater than 0.1 wt % water, such as greater than 0.5 wt %, 0.75 wt %, 1 wt %, 2 wt %, 3 wt %, 4 wt %, or 5 wt % water, preferably greater than 0.5 wt %, more preferably greater than 1 wt %.

In some embodiments, the liquid comprises from 1 wt % to 100 wt % of an oil and/or glycerine, preferably from 5 wt % to 95 wt % of an oil and/or glycerine, more preferably 10 wt % to 90 wt % of an oil and/or glycerine, such as 20 wt % to 80 wt % of an oil and/or glycerine. Alternatively, the liquid comprises from 70 wt % to 100 wt % of an oil and/or glycerine, preferably from 80 wt % to 98 wt % of an oil and/or glycerine, more preferably 85 wt % to 95 wt % of an oil and/or glycerine.

Suitably, the liquid comprises oil. Suitably the liquid comprises at least 1 wt % oil, such as at least 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, or 70 wt % oil. In some embodiments, the liquid comprises less than 100 wt % oil, such as less than 95 wt %, 90 wt %, 85 wt %, 80 wt %, 75 wt %, 70 wt %, 65 wt %, 60 wt %, or 55 wt % oil. Any of the aforementioned lower or upper limits of the ranges may be combined with each other as appropriate, and are herein disclosed.

In some embodiments, the liquid is an oil. In some particular embodiments, the membrane encapsulates from 7 to 20 mL of the liquid, preferably 7 to 18 mL, more preferably 7 to 15 mL, such as 7 to 12 mL or 10 to 15 mL.

Suitably, the liquid comprises glycerin. Suitably the liquid comprises at least 1 wt % glycerine, such as at least 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, or 70 wt % glycerine. In some embodiments, the liquid comprises less than 100 wt % glycerine, such as less than 95 wt %, 90 wt %, 85 wt %, 80 wt %, 75 wt %, 70 wt %, 65 wt %, 60 wt %, or 55 wt % glycerine. Any of the aforementioned lower or upper limits of the ranges may be combined with each other as appropriate, and are herein disclosed.

Suitably, the liquid has a viscosity from 1 to 250,000 cps, preferably 1 to 100,000, more preferably 1 to 20,000 cps, most preferably 100 to 4,000 cps.

Suitably, the liquid has a viscosity of greater than 5 cps, such as greater than 10 cps, 50 cps, 100 cps, 200 cps, 500 cps, 1,000 cps, 1,500 cps, 2,000 cps, 2,500 cps, 3,000 cps, 5,000 cps, 10,000 cps, 15,000 cps, or 20,000 cps. Suitably, the liquid has a viscosity of less than 100,000 cps, such as less than 90,000 cps, 80,000 cps, 70,000 cps, 60,000 cps, 50,000 cps, 40,000 cps, 30,000 cps, 20,000 cps, 15,000 cps, 10,000 cps or 5,000 cps. Any of the aforementioned lower or upper limits of viscosity may be combined with each other, and are herein disclosed.

In some embodiments, the liquid and/or the membrane comprise(s) one or more of an additive, emulsifier, diluent, food additive, food flavouring, fragrance or preservative.

In preferred embodiments, the membrane comprises an additive. The additive provides additional functional/performance effects e.g. increased plasticity, mechanical strength or improved permeability to water. This results in increased stability of the encapsulated liquid and even longer storage times without significant permeation and loss of content. Suitably, the additive is one of or a mixture of two or more of silicone dioxide, kaoline, annatto, bamboo fibre, silk amino acid, chitosan, a starch (such as corn starch or tapioca starch), a hydrocolloid thickener (konjac, guar, cassia, tara, xanthan, locust bean gum, CMC (carboxymethyl cellulose)), 1,3-butylene glycol, acacia, acetic and fatty acid esters of glycerol, acetone, acetylated distarch adipate, acetylated monoglycerides, acid-treated starch, alginic acid, alkaline-treated starch, anoxomer, ascorbic acid, ascorbyl palmitate, ascorbyl stearate, azodicarbonamide, beeswax, bleached starch, bone phosphate, brominated vegetable oil, calcium acetate, calcium alginate, calcium aluminum silicate, calcium ascorbate, calcium benzoate, calcium bromate, calcium carbonates, calcium chloride, calcium citrate, calcium dihydrogen phosphate, calcium disodium ethylenediamine-tetraacetate, calcium DL-malate, calcium ferrocyanide, calcium gluconate, calcium hydrogen sulfite, calcium hydroxide, calcium iodate, calcium lactate, calcium lactate gluconate, calcium lactobionate, calcium peroxide, calcium phosphate, calcium polyphosphates, calcium propionate, calcium pyrophosphate, calcium salts of fatty acids, calcium silicate, calcium sorbate, calcium stearate, calcium stearoyl lactylate, calcium sulfate, calcium tartrate, calciumiodiate, candelilla wax, carbamide, carbon dioxide, carnauba wax, carob bean gum, castor oil, cellulose gum, choline salts and esters, citric acid, citric and fatty acid esters of glycerol, crosslinked sodium carboxymethylcellulose, cupric sulfate, D-alpha-tocopherol, dammar gum, decanoic acid, dedesoxycholic acid, dedextrins, dextrin ethyl cellulose, dextrose, diacetyltartaric acid esters of mono- and diglycerides of fatty acids, diammonium hydrogen phosphate, dicalcium pyrophosphate, diethyl pyrocarbonate, ethyl alcohol, ethyl cellulose, ethyl hydroxyethyl cellulose, ethyl p-hydroxybenzoate, ethyl protocatechuate, ethylene dichloride, esters of glycerol and thermally oxidized soy bean fatty acids, ethoxylated mono- and diglycerides, ethyl hydroxyethyl cellulose, formic acid, gellan gum, gelatin, genipin, gibberellic acid, glucono delta-lactone, glycerin, glycerol, glycerol ester of wood rosin, guaiac resin, guar gum, gum acacia, gum arabic, gum ghatti, gum guaiac, heptylparaben, peroxide derivatives, hydrogen peroxide, hydroxylated lecithin, hydroxypropyl cellulose, hydroxypropyl distarch. In preferred embodiments, the additive is one or a mixture of two or more of kaoline, silk amino acid, silicone dioxide, chitosan. In further preferred embodiments, the additive comprises a food grade or natural bioderived additive such as high or low methoxyl pectin (LMP/HMP), dextrose, fructose, natural clays with a particle size less than 30 microns (preferably between 5-10 microns), polyphenols such as tannic or gallic acid, seaweed derived proteins or extracts such as fucoidans, proteins such as zein, chitosan. The additives may also comprise waste materials and extracts derived from seaweed extraction processes, such as waste seaweed fibers, filtration agents or fruit and vegetable peels, including those with high polyphenol content such as grapes, honey, mango, blueberries, pomegranates, or apple. In one embodiment, the additive is a starch.

Common food emulsifiers that may be included are mono and diglycerides, polysorbates, guar gum, canola oil, soy and egg lecithin, mustard, stearoyl lactylates, sorbitan esters, polyglycerol esters, sucrose esters and lecithin.

Common natural cosmetic emulsifiers that may be included are waxes such as candelilla wax and beeswax, cetearyl Alcohol and Cetearyl Glucoside, cetearyl wheat straw glycosides and cetearyl alcohol, sorbitan Olivate, cetearyl Olivate.

In some embodiments, a filler additive is incorporated for improvement to permeability performance or a desirable texture aesthetic when the product is formed. Natural waste products can be incorporated into the product through the film/membrane. The benefit of this is that it can reduce the amount of raw materials needed in the initial formulation and to reduce carbon footprint. It can also improve the performance of the material e.g. improvement to permeability, water solubility, heat sealability or overall compression strength of the final product. Examples include seaweed waste fibers, citrus fibers, bamboo fibers, starch fibers, natural clays or any other fiber derived from a waste source. Ideally the inserted fillers or fibers have a small particle size less than 50 microns.

In some embodiments, the membrane comprises an additive to improve compression strength of the membrane, such as konjac glucommanan or a sugar alcohol (such as mannitol, xylitol, sorbitol). Preferably, the sugar alcohol is mannitol, as it offers the most improvement to the membrane.

In some embodiments, the membrane comprises an additive to improve permeability and brightness, such as low methoxy pectin.

The membrane mixture may be blended with a plasticising additive in order to increase plasticity prior to pumping the solution through a die to create ribbons or films. Therefore, in some embodiments, the additive comprises a plasticising additive. Suitably, the plasticising additives may comprise glycerol, sorbitol, sodium stearate, agar, carrageenan or pectin, or a mixture of two or more thereof, preferably glycerol, sorbitol, or a mixture of two or more thereof. In addition, pectin can be added to make the membrane wall of the film pod visually clearer and brighter. The same effect can be observed with the addition of other simple sugars such as fructose or dextrose which can also be added to improve permeability of the membrane. Therefore, in some embodiments, the membrane further comprises pectin or a simple sugar such as fructose or dextrose, or a mixture of two or more thereof.

Examples of other plasticisers include Dihydric acids (Ethylene glycol, propylene glycol), Trihydric alcohols (Glycerol, polyglycerol), Sugar alcohols (Sorbitol, mannitol, maltitol, xylitol), Saccharification products of reduced, starch (glucose, fructose, galactose andxylose), Disaccharides (saccharose, maltose and lactose), Amine acids (in combination with polyols e.g. Proline, glycine, anilin), Fatty acids-short chains (Capric acid, caproic acid).

In some embodiments, the membrane comprises a thickener to improve processability, mechanical strength and permeability.

Suitably, the thickener comprises a natural or vegetable gum, such as a natural polysaccharide from the glucomannan group or a thickener derived from seaweed.

In some embodiments, the thickener comprises one of maltodextrin, starch, modified starch, cellulose gel, guar gum, tara gum, gum tragacanth, locust bean gum, microcrystalline cellulose, pectin, gellan gum, glucumannans, succinoglucan, konjac glucomannan, xanthan gum, mesquite gum, gum arabica, glycerol, low/high methoxyl pectin or mixtures of two or more thereof.

In preferred embodiments, the thickener comprises maltodextrin, glycerol, guar gum, pectin, xanthan gum, starch (such as modified food starch, particularly cornstarch) low/high methoxyl pectin, or konjac glucommanan or a mixture of two or more thereof. Most preferably, the thickening agent comprises starch, and/or glycerol and/or maltodextrin.

The membrane may also include a natural or bioderived surfactant to enhance or reduce elasticity of the membrane as required. Surfactants can also be used as a wetting agent to increase or decrease the interfacial surface tension of the membrane mixture and different content materials, or as an emulsifier to enable the incorporation of hydrophobic ingredients into the membrane or contents.

Hydrophile Lipophile Balance (HLB) is a way of measuring a substances solubility within water or oil. The HLB characterisation system is used to identify the correct type of surfactant for a given application. The HLB system enables the user to assign a HLB value to a surfactant and a HLB requirement to the application for that surfactant. Properly matching these values helps develop appropriate formulations for each application. For example, high HLB surfactants can be used to increase hydrophilicity of a membrane mixture and to stabilise oils (when incorporated into the membrane or the contents). Mid HLB surfactants above 10 e.g. sorbitan esters are more hydrophilic.

Anionic surfactants may improve elasticity in the membrane. Therefore, in some embodiments, the membrane comprises one or a mixture of two or more anionic surfactants such as sodium stearate or sodium lauryl sulfate.

Nonionic surfactants may reduce elasticity. Therefore, in some embodiments, the membrane comprises one or a mixture of two or more nonionic surfactants, such as lauryl glucoside, decyl glucoside, glycol distearate, glycerol monostearate, propylene glycol isostearate, a Tween® (such as Tween® 20, 40, 60, 80), a Span™ (such as Span™ 20, 40, 60, 80, 85) or a Polysorb® (such as Polysorb® 85/70/00), polysorbate (such as polysorbate 20, 40, 60, 80) or lecithin.

In some embodiments, the additive, thickener, diluent or preservative that may be present are selected to have a neutral odour and/or taste. Using a mixture with neutral taste and/or odour has the advantage of avoiding taste contamination.

The addition of a food additive, food flavouring or fragrance to the liquid and/or the membrane may be for the purposes of creating a more desirable consumer product, for example to enhance the taste or smell of it. Suitable food additives, food flavourings and fragrances include natural occurring sugars such as dextrose or fructose.

A preservative is a substance or a chemical that is added to products such as food products, beverages, cosmetics, and many other products to prevent decomposition by microbial growth or by undesirable chemical changes. Suitable preservatives include a mixture of citric acid and potassium sorbate, a mixture of lactic acid and potassium sorbate, or a mixture of ascorbic acid and potassium sorbate. In preferred embodiments, the preservative comprises citric acid and potassium sorbate, as this mixture has the least effect on flavour and cost.

Diluents may be used to reduce the viscosity of the liquid. Suitable diluents include plasticisers such as glycerol or propylene glycol.

In some embodiments, the liquid and/or the membrane are edible or suitable for applying onto a body part.

Suitably, the liquid may be edible and comprise, such as consist of, various food and drink related products. In accordance with the disclosure herein, the skilled person will understand that only products that have a water content of less than 20 wt % are suitable. Suitably the liquid comprises greater than 80 wt % of a food or drink product, preferably greater than 85 wt %, preferably greater than 90 wt %, more preferably greater than 95 wt %, such as greater than 98 wt %, most preferably 100 wt %. Suitable food and drink related products are herein described below (but not limited to):

Sauces and condiments, which are shear-thinning non-Newtonian fluid food compounds typically comprising salt, sugar and one or more spices which when added to a food enhances the flavour of the food. In some embodiments, the food or drink product is a sauce or condiment.

Energy gels are a class of low water content carbohydrate rich gels, typically comprising a blend of sugars, most often maltodextrin and fructose, caffeine and preservatives. In some embodiments, the food or drink product is an energy gel.

In some embodiments, the food or drink product is an edible oil, such as extra virgin olive oil, chilli oil, garlic oil, rapeseed oil, sesame oil, coconut oil, vegetable oil, sunflower oil, safflower, palm kernel oil, palm oil, canola oil, peanut oil, wheat germ oil, walnut oil, soybean oil, corn oil, almond oil, hazelnut oil, avocado oil, or ghee. Preferably, the food or drink product is an olive oil, flavouring oil such as chilli or garlic oil or a speciality oil such as sesame or coconut oil.

In some embodiments, the food or drink product is a condiment such as mayonnaise, ketchup, mustard, yogurt, cream cheese.

Suitably, the liquid may be non-edible and may comprise, such as consist of, personal hygiene and cosmetic products. In accordance with the disclosure herein, the skilled person will understand that only products that have a water content of less than 20 wt % are suitable.

Personal hygiene and cosmetic products are constituted mixtures of chemical compounds derived from either natural sources or synthetically created ones, designed for personal care and skin care, and can be used to cleanse or protect the body or skin. Suitably the liquid comprises greater than 80 wt % of a personal hygiene or cosmetic product, preferably greater than 85 wt %, preferably greater than 90 wt %, more preferably greater than 95 wt %, such as greater than 98 wt %, most preferably 100 wt %.

Personal hygiene and cosmetic products suitable for use in the present invention include oil or emulsion based cleansers, toners, serums, moisturisers, balms, shampoo, conditioner, shower gel, skin creams, face masks, exfoliants, moisturisers, sun creams, hair conditioner, hair gel, hair cream, soap, liquid soap, hand soap, body wash, lip-gloss, foundation, lipstick, eyeshadow and personal care oil contents. Personal care oils include lavender oil, jojoba oil, CBD oil, petroleum jelly, grapeseed, clay oil, essential oils and rose hip oils. Preferably the personal hygiene and cosmetic product is one of shower gel, shampoo, conditioner, oil based soap, sun cream, skin care, cream, toothpaste, or personal care oil.

Shower gel, shampoo, conditioner and liquid soap are liquids used for cosmetic and hygiene applications typically comprising surfactants, foaming agents, conditioners, thickeners, opacifiers, sequestering agents, preservatives, special additives, and/or fragrance. In some embodiments, the personal hygiene and cosmetic product is an oil based soap, an oil based cleanser or soap, shower gel, shampoo or conditioner.

Sun creams comprise an active ingredient intended to protect the skin from the sun. In some embodiments, the personal hygiene and cosmetic product is sun cream.

Toothpaste is a paste or gel dentifrice typically comprising glycerine to stop the paste from drying out, calcium carbonate which acts as an abrasive, sodium lauryl sulfate which acts as a detergent and makes toothpaste foam, sodium saccharin for flavour, and fluoride to strengthen tooth enamel. In some embodiments, the personal hygiene and cosmetic product is toothpaste.

In some embodiments, the length of the soft gel capsule is from 50 mm to 80 mm, preferably 55 mm to 75 mm, more preferably 60 mm to 70 mm. The length of the soft gel capsule is considered as the longest measurement. Suitably the width of the soft gel capsule is from 5 mm to 25 mm, preferably 10 to 20 mm.

In some embodiments, the volume of the liquid encapsulated in the membrane is from 0.1 mL to 40 mL, preferably 0.1 mL to 30 mL, more preferably, 0.1 to 20 mL, yet more preferably from 0.1 to 15 mL, preferably 7 to 12 mL. Suitably the volume is 7 to 20 mL, such as 7 to 15 mL, such as 10 to 14 mL. Preferably, in such embodiments, the membrane comprises an extract derivable from Euchema Spinosum, even more preferably carrageenan.

In some embodiments, the volume of the liquid encapsulated in the membrane is from 2 to 30 ml of the liquid, preferably from 2 to 20 mL, more preferably from 2 to 10 mL. Preferably, in such embodiments, the membrane comprises an extract derivable from Gelidium, Gracilaria, or a cellulose extract derivable from seaweed, even more preferably agar and HPMC.

In some embodiments, the volume of the liquid encapsulated in the membrane is at least 0.1 mL, such as at least 0.25, mL, 0.5 mL, 0.75 mL, 1 mL, 1.5 mL, 2 mL, 3 mL, 4 mL, 5 mL, 6 mL, 7 mL, 8 mL or 9 mL. In some embodiments, the volume of the liquid encapsulated in the membrane is less than 40 mL, such as less than 35 mL, 30 mL, 25 mL, 20 mL, 15 mL, 12 mL, or 10 mL. Any of the aforementioned lower or upper limits of the ranges may be combined with each other, and are herein disclosed.

In some embodiments, the liquid is a single dose. A single dose means a single portion usually for use by a single consumer, for example an amount of toothpaste suitable for a single toothbrushing, or an amount of moisturiser suitable for a single application to the user's face, or a single portion of a sauce, such as mayonnaise.

The single dose has the advantage of reduced waste and reduced plastic pollution as it would enable consumers to use the appropriate amount of a product (e.g. toothpaste portion or a condiment portion, replacing the individual sachets made of non-biodegradable plastic that are used in many food outlets globally). Additionally, it would enable consumers to only carry with them the appropriate amount of product when traveling, e.g. two pearls of toothpaste for a day of travelling, instead of carrying a whole plastic toothpaste tube, which is generally also not recyclable.

It is an advantage of the invention that the liquid remains stably stored within the membrane. This allows the product contained within the membrane to be stored over a significant period of time. In some cases the membrane may have a shelf life of up to 2 years without the liquid degrading or leaking out of the membrane, depending on the seaweed extracts used and the properties of the content.

The membrane contains the liquid without losing significant amounts of it via permeation. Suitably, the liquid has negligible mass loss 24 hours after manufacturing, preferably less than 1 wt % mass loss 24 hours after manufacturing, as measured by the method described in Example 3.

In some embodiments, the membrane is in the form of a soft gel capsule or a film pod. In some embodiments, the membrane is spherical, ovoid, droplet or pipette shape.

In some embodiments, several soft gel capsules/film pods are grouped together such as within a second membrane. The second membrane may be a further membrane comprising an extract derivable from seaweed, or other suitable hydrogel or protective layer which is peelable or otherwise removable from the product but provides further hygienic protection to the encapsulated product. Such a layer would be similar to that of a fruit skin and designed to allow the consumer to remove it from the product as required. This layer may comprise a wax or fatty acid composition, or a mixture of hydrophobic materials that may act as a further barrier to water permeation. Examples of waxes include beeswax, candelilla wax, carnauba wax, jojoba oil, ouricury wax. Examples of fatty acids include capric acid, lauric acid, myristic acid, palmitic acid or stearic acid. Natural sources of these fatty acids include coconut oil, palm kernel oil, oil palms, coca butter, shea butter.

The exterior of the encapsulated product, comprising an extract derivable from seaweed, whether it comprises a further secondary layer or not, may be a useful substrate to which edible ink or imagery may be applied in the form of text, pictures or logos. Text of a logo can be printed or applied for marketing purposes or hidden and introduced between two layers in the case of a secondary membrane.

In a second aspect, the invention provides a method of preparing the liquid encapsulated within a membrane according to the first aspect, said method comprising the steps of:
(i) providing a solution of an extract derivable from seaweed;
(ii) pumping the solution of step (i) through a die to create two ribbons or films of gel;
(iii) injecting a liquid having a water content of less than 20 wt % between the two ribbons or films;
(iv) sealing the two ribbons or films around the liquid thereby forming the liquid encapsulated within a membrane.

The second aspect of the invention may have any of the features described above for the first aspect.

In some embodiments, the concentration of the extract derivable from seaweed in the solution of step (i) is present in an amount of from 8 wt % to 20 wt %, preferably from 8 wt % to 15 wt %, more preferably 8 wt % to 13 wt %, more preferably 8 wt % to 10 wt %, yet more preferably 2 wt % to 10 wt %, even more preferably 2 wt % to 8 wt %, and even more preferably 2 wt % to 5 wt %.

In some embodiments, the concentration of the extract derivable from seaweed in the solution of step (i) is present in an amount greater than 1 wt %, such as greater than 2 wt %, or 3 wt %. In some embodiments, the concentration of the extract derivable from seaweed in the solution of step (i) is present in an amount of less than 25 wt %, such as less than 24 wt %, 23 wt %, 22 wt %, 21 wt %, 20 wt %, 19 wt %, 18 wt %, 17 wt %, 16 wt %, 15 wt %, 14 wt %, 13 wt %, 12 wt %, 11 wt %, 10 wt %, 9 wt %, 8 wt %, 7 wt %, 6 wt %, 5 wt %, or 4 wt %. Any of the aforementioned lower or upper limits of the extract derivable from seaweed concentration may be combined with each other, and are herein disclosed.

In some embodiments, wherein extract derivable from seaweed is a cellulose polymer, the concentration of said cellulose polymer in the solution of step (i) is present in an amount from 10 wt % to 20 wt %, preferably from 10 wt % to 15 wt %, more preferably 10 wt % to 13 wt %.

In some embodiments, the film obtained in step (ii) is fed over a rotating drum covered in cavities which the film may be vacuum formed into. In one embodiment, the cavities are hemispherical.

In some embodiments, the injection of liquid in step (iii) is done using a syringe (manual or machine operated) or a pump (such as a peristaltic pump) or other injecting device.

Suitably, the liquid is provided as a predetermined dosage.

In some embodiments, during the sealing of the film pod in step (iv), a die may simultaneously cut and eject the film pod from excess film.

In some embodiments, the method may comprise a further step of applying a second membrane around the membrane of the encapsulated liquid. The second membrane may also comprise an extract derivable from seaweed. The second membrane reduces the water permeability.

Additives can be added to the membrane to provide additional functional/performance effects e.g. increased mechanical strength or improved permeability to water. Thus suitably the solution in step (i) further comprises an additive as described earlier in the specification, and/or a thickening agent, and/or stabilizer and/or emulsifier. Preferably the additive and/or a thickening agent, and/or stabilizer and/or emulsifier comprises one or more of glycerol, ECOTween 80, polysorbate 80, modified starch, propylene glycol, preferably ECOTween 80/polysorbate 80 and/or modified starch and/or propylene glycol.

In some embodiments, the additive comprises a modified starch in 5 to 50 wt %, preferably 10 to 40 wt %, more preferably 20 to 30 wt % with respect to the weight of the solution in step (i). Preferably, in such embodiments, the solution in step (i) comprises carrageenan.

Alternatively, the additive may comprise modified starch in 1 to 10 wt %, preferably 1 to 5 wt %, more preferably 1 to 2 wt % with respect to the weight of the solution in step (i). Preferably, in such embodiments, the solution in step (i) comprises HPMC.

In some embodiments, the additive comprises glycerin in 5 to 50 wt %, preferably 10 to 40 wt %, more preferably 15 to 30 wt % with respect to the weight of the solution in step (i). Preferably, in such embodiments, the solution in step (i) comprises carrageenan.

Alternatively, the additive comprises glycerin in 1 to 10 wt %, preferably 1 to 5 wt %, more preferably 1 to 3 wt %, yet more preferably 1 to 2 wt % with respect to the weight of the solution in step (i). Preferably, in such embodiments, the solution in step (i) comprises agar or HPMC.

In some embodiments, the additive comprises ECOTween 80 or polysorbate 80 in 0.01 to 0.5 wt %, preferably 0.01 to 0.1 wt %, more preferably 0.01 to 0.05 wt % with respect to the weight of the solution in step (i). Preferably, in such embodiments, the solution in step (i) comprises agar.

Alternatively, the additive comprises propylene glycol 1 to 10 wt %, preferably 1 to 5 wt %, more preferably 2 to 3 wt % with respect to the weight of the solution in step (i). Preferably, in such embodiments, the solution in step (i) comprises HPMC.

Additives can be added to the membrane to provide additional functional/performance effects e.g. increased mechanical strength or improved permeability to water. Thus suitably the solution in step (i) further comprises an additive as described previously, such as silicone dioxide, kaoline, annatto, bamboo fibre, silk amino acid, or chitosan.

Suitably, the solution in step (i) may be used to made films, such as by solvent casting then forming and moulding into a desired final product shape. Suitably, the shape is designed for a single use, such as olive oil capsule, detergent pod or a shampoo sachet.

Solvent-casting is a well-known film-making process. A solution of polymers, plasticizers, and any other ingredients are dissolved in a volatile solvent, like water or ethanol. This material, referred to as the film dope, is spread out using classical solvent-cast film deposition methods onto a continuous roll of release media, such as a steel belt, silicone belt or plastic coated substrate. The coated media is passed through a drying apparatus, such as an oven or a convection chamber, to drive off the solvents. The dried film is then die cut into strips and packaged individually.

Pods, capsules and sachets can be formed using any known machinery, including liquid or dry laundry pod machines.

Sachets can be made using a vertical form fill seal machine, also known as a VFFS. This is a common bagging machine used to package goods into bags as part of a production line. It can be used to make sachets with a variety of different types of sealing e.g. back, front, side. The VFFS can be designed to work with powders, granules, liquids or sauces. Generally they are automatic or semi-automatic.

Sachets can be made to any desired width and length, preferentially for sampling or unidose sachets a width of 50 mm is used with adjustable lengths. Back sealing is the preferred type of seal. For sealing, the film needs to be able to seal under heat in under 5 seconds. Therefore, in some embodiments, the film is able to seal under heat in under 5 seconds, preferably in under 1 second. In alternative preferred embodiments, the film is able to seal under heat in between 1 to 3 seconds. Temperatures in the range of 150-210° C. may be used.

According to some embodiments, novel product shapes such as capsules in the shape of pipettes and drops (e.g. as shown in FIG. 4) may be created using the rotary die method.

The film or membrane material may be completely microplastic free, naturally biodegradable and home compostable. It normally takes less than six weeks for packaging according to the present invention to completely decompose in a home compostable environment. In contrast, PLA and other bioplastics require specific recycling pathways and specialised industrial composting equipment to degrade.

The final product shape may be made by heat sealing e.g. to mould the film around a specific shape to create a pod or pipette, or folded to form a sachet. The material can also be optionally water soluble at specific temperatures or under specific conditions for use. In some embodiments, the product (e.g. pod or sachet) can dissolve in under 6 seconds, preferably in under 3 seconds, leaving no trace in the liquid. e.g. sachet that dissolves in hot water to release the contents such as coffee or dried soup into a vessel, or dissolves in cold-warm water for detergent or laundry applications.

A variety of colours and textures can be achieved through a combination of ingredient choice and processing. Any type of naturally derived pigment can be added to the formulation to impart a colour in the final product e.g. spirulina for green/blue, charcoal for black. Addition of waste fibers e.g. bamboo, citrus, seaweed fibers can also impart a natural colour or texture to the film.

In addition, through the process of solvent casting, using a film or tape casting machine, the substrate onto which the film is created can be used to imprint a texture on the film that may be desirable in some instances, e.g. textured lines or cross hatching.

Suitably, the products according to the present invention are composed of naturally derived ingredients, preferably vegan and edible.

The thickness of the film or membrane may vary depending on the end target application. The thickness may range between 20 microns to 100 microns, preferably between 20 to 60 microns, more preferably 20 to 40 microns, such as 45 microns. For applications such as containing screws or other hard items such as stationary, a thickness of 40 to 60 microns is preferred.

The films of the present invention are compatible with traditional packaging machinery for the formation of different product shapes with different content types (aqueous and non-aqueous, powders and liquids). The films can be made to suit specific packaging machinery for the formation of pods, sachets or pipettes as the contents require.

Natural waste products can be incorporated into the product through the film/membrane. The benefit of this is that it can reduce the amount of raw materials needed in the initial formulation and to reduce carbon footprint. It can also improve the performance of the material e.g. improvement to permeability, water solubility, heat sealability or overall compression strength of the final product.

Branding can be achieved through a selection of available colours, opacities, and textures. As well as direct printing onto the film using various inks, including food safe options.

In a third aspect, the invention provides the use of the liquid encapsulated within a membrane according to the first aspect of the invention or the container according to the fourth aspect to:
(i) package and/or store the liquid; and/or
(ii) transport the liquid; and/or
(iii) provide a single dose of the liquid to a user.

The third aspect of the invention may have any of the features described above for the first and second aspects of the invention.

In a fourth aspect, the invention provides a container comprising (such as consisting of) at least one liquid encapsulated within a membrane according to the first aspect of the invention. The container acts as secondary packaging for storing and transporting the capsules of the invention.

The container may be made of a reusable or recyclable material. The container may be a glass jar, or a cardboard box or tube.

The liquid encapsulated in a membrane of the first aspect of the invention may be packaged in a container for storage or transport, without the need to submerge the capsules in liquid. Accordingly, in some embodiments, the container does not comprise further liquid.

The invention will now be described by way of the following non-limiting examples.

The skilled person will understand that features which are optional can be used in different combinations to construct various different embodiments and examples of the invention not limited to those shown herein.

### EXAMPLES

### Example 1—Preparation of Gelidium Based Seaweed Extract Film Packaging

### Method of Preparation

A Gelidium based seaweed extract as agar or Gelidium gel solution was prepared in which the concentration of the seaweed extract was within the range of 2 to 5%, glycerol within the range of 1 to 3% and ECO Tween 80 within the range of 0.01 to 0.05% by weight of the solution (the seaweed extract was stirred in a heated solution of glycerol, ECO Tween 80 and water whilst being stirred under vacuum). The hot seaweed extract gel solution was cast into a film which was then oven heated and air dried until fully dry.

The film was fed over a rotating drum covered in hemispherical cavities which the film was vacuum formed into. A predetermined volume of cosmetic content (gel comprising less than 20 wt % of water, shampoo with a water content of 12.6%, or conditioner with a water content of 9.6%) in the range of 2 to 20 ml was dispensed into the cavities described above via a fill injector.

A second film was sealed on a roller to the original film described above with the application of heat and pressure created a sealed content filled agar film capsule. During the sealing of the filled capsules, the die simultaneously cut and ejected the capsules from the excess film sheet.

The same process was also used to make film pods containing toothpaste (water content 5-10%), sauces/condiments (ketchup water content 15-20% and mayonnaise water content 6-10%), energy gel (water content 10-15%), and coffee (5-15% in solution), each comprising less than 20 wt % of water. The contents remained stably within the film pods for at least 3 months.

The same film can be also fed over a vertical or horizontal form-fill-seal machine. A predetermined volume of (each comprising less than 20 wt % of water) content in the range of 2 to 20 ml was dispensed into the formed sachet packaging.

### Example 2—Preparation of Cellulose Extract Film Packaging

### Method of Preparation

A seaweed derived cellulose extract (HPMC) gel solution was prepared in which the concentration of seaweed derived cellulose was 10-13%, starch within the range of 1 to 2%, glycerol within the range of 1 to 2% and propylene glycol within the range of 2 to 3% by weight of the solution (the seaweed derived cellulose and starch were stirred in a heated solution of propylene glycol, glycerol and water whilst being stirred under vacuum). The seaweed derived cellulose gel solution was then cast into a film which was oven heated and air dried till fully dry.

The film was fed over a rotating drum of a rotary pod machine covered in hemispherical cavities which the film is vacuum formed into. A predetermined volume of condiment/sauce (each comprising less than 20 wt % of water) content in the range of 2 to 20 ml was dispensed into the cavities described via a fill injector.

A second film was sealed on a roller to the original film described above with the application of heat and pressure creates a sealed content filled seaweed derived cellulose film capsule. During the sealing of the filled capsules the die simultaneously cut and ejected the capsules from the excess film sheet.

The same process was also used to make film pods containing cosmetic products, toothpaste (water content 5-10%), sauces (ketchup water content 15-20% and mayonnaise water content 6-10%), energy gel (water content 10-15%), and coffee (5-15% in solution), i.e. each comprising less than 20 wt % of water. The contents remained stably within the film pods for at least 3 months.

The same film can be also fed over a vertical or horizontal form-fill-seal machine. A predetermined volume of (each comprising less than 20 wt % of water) content in the range of 2 to 20 ml was dispensed into the formed sachet packaging.

### Example 3—Preparation of a Euchema Spinosum Packaging

### Method of Preparation

A seaweed extract from Euchema Spinosum (carrageenan) based gel solution was prepared in which the concentration of seaweed extract was within the range 8% to 10%, modified starch within the range of 20 to 30% and glycerin within the range of 15 to 30% by weight of the solution (powdered Euchema Spinosum extract and modified starch were added while stirring under vacuum in a heated solution of glycerin and water).

The gel formulation was pumped through machine dies creating two ribbons of gel. The flat gel ribbons were cut with two rotating die cylinders into discs of predetermined size and shape.

A set volume of olive oil (i.e. 100% oil and thus 0% water content) in the range 0.1-15 ml was measured and dispensed by a pump between the cut discs as the gel ribbons described above came together towards the fill injector

The filled capsule halves were then sealed by the application of heat and pressure as the die assembly rotated and the two dies came together. The ejection of sealed capsules occurred simultaneously during the sealing movement. The capsules were dried and packed.

The same process was also used to make soft gel capsules containing a cosmetic product, toothpaste, sauces, and condiments, each comprising less than 20 wt % of water.

### Assessment of Permeability

The following method was used to determine permeability, as a wt % mass loss over time. The capsule was placed in a storage jar with an airtight lid, and the jar with capsule inside was then weighed, recording this as the initial mass. The jar was then stored in the fridge, or room temperature if performing ambient temperature permeability. The weight was then measured after a period of time, e.g. 24 hours, 48 hours, 72 hours and 96 hours by removing the jar from its storage location, opening the lid, drying any excess moisture from the capsule surface or from inside the jar and then subsequently weighing the capsule inside the jar. The resulting mass difference can then be reported as a % mass loss over time, and acts as an indication of the capsule permeability performance.

TABLE 1
Permeability assessment of carrageenan capsule

| | Mass loss over |
|---|---|
| Capsule content composition | 24 hrs (Permeability) |
| Chilli oil (0 wt % water content) | Negligible <1% |
| Olive oil (0 wt % water content) | Negligible <1% |
| Garlic oil (0 wt % water content) | Negligible <1% |
| Coconut oil (0 wt % water content) | Negligible <1% |
| Mayonnaise (7-8 wt % water content) | Minimal <5% |

Table 1 shows that contents with a water content of less than 20 wt % stably remained within the capsules, contrary to those with a water content of greater than 20 wt %.

### Example 4—Preparation of Gelidium Based Films

A film was produced using a HED Solvent casting machine TC-71LC. The wet casting thickness was pre-determined to be 400-800 μm

The film mixture was prepared using a Gelidium based seaweed extract (agar) (5-6%), glycerol (2-4%), polysorbate 80 (0.01%-0.05%), water (90-92%), and integrated waste fibers (bamboo fibers 0.02%-0.2%)

The heat seal parameters were set at 170° C., 1 second dwell time.

The film was stable in ambient and relative humidity. The moisture content of the film was 10-15% at the time of filling.

The contents tested with the film thus obtained include washing gel, bath soap, lip gloss, sunscreen, facial oils, cleansing oils, Vaseline, hair oil, all of which had less than 20 wt % water content. All of these liquids incorporated into the film were stable for at least 3 months.

Other contents with water content of greater than 20 wt % such as fabric softener, contact lenses lotion did not result in a stable product when encapsulated in the film, as water was observed to leak out over time.

### Example 5—Preparation of HPMC Films

A water soluble film was prepared using a mixture of HPMC, maltodextrin, waxy rice starch, propylene glycol, glycerol and water. The method is the same as for the Gelidium (agar) film in Example 4.

This film had an average thickness of 40 μm. Moisture content of the film was 10-15% (at the time of filling).

Sachets were made from a flat sheet of film, measuring 80×100 mm, and sealed using an impulse heat sealer (170° C., 2 s heating time, 8 s cooling time). Between 7.0-10.0 g of formulations (detergent containing 0 wt %, 2.5 wt %, 5 wt %, 7.5 wt %, 10 wt %, 12.5 wt %, 15 wt % water) was added to each pouch.

Samples were then initially kept in separate sealed glass jars, to avoid drying out of the samples, as this initial testing film did not provide a complete moisture barrier.

After 24 hours, samples with 0.0%-10.0% solutions inside were dry to the touch, and no indication that the internal content was impacting the film. The samples containing 12.5% water were ever so slightly tacky although the solutions were still contained within the film. The samples containing 15.0% water felt slightly more tacky than the samples containing 12.5 wt % water, and were cool to the touch, indicating that a little water had been taken up by the film.

### CONCLUSION

Various liquids with a water content of less than 20 wt % have been successfully prepared in various sizes, including sizes appropriate for single dose use. Both edible and non-edible products within the capsules have been made and have been found to be stable over time. Thus they are useful for long-term packaging and storage of consumer products. Advantageously, the film pods and soft gel capsules as disclosed herein offer an environmentally friendly alternative to the currently widely used single-use and non-biodegradable or non-recyclable plastic packaging. This can help the ever increasing issues relating to climate change and environmental damage caused in part by plastic packaging.

1. A liquid encapsulated within a membrane, wherein the liquid has a water content of at least 1 wt % and less than 20 wt %, wherein the membrane comprises an extract derivable from seaweed, wherein the membrane or liquid comprises one or more of an additive, wherein the liquid comprises
i) a personal hygiene or cosmetic product; or
ii) a food or drink product.

2. The liquid encapsulated within a membrane as claimed in claim 1, wherein the liquid is water-based or glycerine-based.

3. The liquid encapsulated within a membrane as claimed in claim 1, wherein the liquid has a water content of less than 15 wt %, preferably less than 13 wt %, more preferably less than 10 wt %.

4. The liquid encapsulated within a membrane as claimed in claim 1, wherein the extract derivable from seaweed is sourced from one of Gelidium, Gracilaria, Chondrus crispis, Eucheuma Denticulatum (Spinosum), Kappaphycus Alvarezii (Cottonii), Gigartina skottsbergii, Euchema cottonii, Gigartina stellata, Ulva or Chondrus.

5. The liquid encapsulated within a membrane as claimed in claim 1, wherein the extract derivable from seaweed is carrageenan, agar, or a cellulose polymer, such as hemicellulose, microfibrilated cellulose, microcrystalline cellulose, Hydroxypropylmethyl Cellulose (HPMC) or Carboxymethyl cellulose (CMC.

6. (canceled)

7. The liquid encapsulated within a membrane as claimed in claim 1, wherein the membrane or liquid comprises one or more of an additive, emulsifier, diluent, carrier, food additive, food flavouring, fragrance, or a preservative, preferably an additive.

8. The liquid encapsulated within a membrane as claimed in claim 1, wherein the additive is silicone dioxide, kaoline, annatto, bamboo fibre, silk amino acid, chitosan, a plasticiser such as glycerin or sorbitol, a starch such as cornstarch or tapioca, or a hydrocolloid thickener.

9. The liquid encapsulated within a membrane as claimed in claim 1, wherein the liquid is edible and/or suitable for applying onto a body part.

10. The liquid encapsulated within a membrane as claimed in claim 1, wherein the personal hygiene or cosmetic product is shower gel, shampoo, conditioner, sun cream, skin care cream, or toothpaste.

11. The liquid encapsulated within a membrane as claimed in claim 1, wherein the food or drink product is a sauce, condiment, energy gel or coffee.

12. The liquid encapsulated within a membrane as claimed in claim 1, wherein the membrane encapsulates:
(i) from 0.1 to 30 mL of the liquid, preferably from 0.1 to 20 mL, more preferably from 0.1 to 15 mL; or
(ii) from 2 to 30 mL of the liquid, preferably from 2 to 20 mL, more preferably from 2 to 10 mL.

13. The liquid encapsulated within a membrane as claimed in claim 1, wherein the liquid is a single dose.

14. The liquid encapsulated within a membrane as claimed in claim 1, wherein the liquid remains stably stored within the membrane for at least 1 week, preferably at least 2 weeks, more preferably at least 3 weeks, yet more preferably at least 4 weeks, wherein the liquid has less than 1 wt % mass loss after 24 hours.

15. (canceled)

16. The liquid encapsulated within a membrane as claimed in claim 1, wherein the membrane is in the form of a soft gel capsule or a film.

17. The liquid encapsulated within a membrane as claimed in claim 1, wherein the liquid comprises from 1 wt % to 100 wt % of glycerine, preferably from 5 wt % to 95 wt % of glycerine, more preferably 10 wt % to 90 wt % of glycerine, such as 20 wt % to 80 wt % of glycerine.

18. (canceled)

19. A liquid encapsulated within a membrane, wherein the liquid is an oil, wherein the membrane comprises an extract derivable from seaweed and wherein the volume of the liquid encapsulated in the membrane is from 7 to 20 mL, 7 to 15 mL, or preferably 7 to 12 mL.

20. (canceled)

21. (canceled)

22. The liquid encapsulated within a membrane as claimed in claim 1, wherein the membrane is spherical, ovoid, droplet or pipette shape.

23. A method of preparing the liquid encapsulated within a membrane as claimed in claim 1, said method comprising the steps of:
(i) providing a solution of an extract derivable from seaweed;
(ii) pumping the solution of step (i) through a die to create two ribbons or films of gel;
(iii) injecting a liquid having a water content of less than 20 wt % between the two ribbons or films of gel;
(iv) sealing the two ribbons or films around the liquid thereby forming the liquid encapsulated within a membrane.

24. The method of claim 23, wherein the concentration of the extract derivable from seaweed in the solution of step (i) is present in an amount:
(i) from 8 wt % to 20 wt %, preferably from 8 wt % to 15 wt %, more preferably 8 wt % to 13 wt %, such as 8 wt % to 10 wt %; or
(ii) from 2 wt % to 10 wt %, preferably from 2 wt % to 8 wt %, more preferably 2 wt % to 5 wt %.

25. The method of claim 23, wherein the extract derivable from seaweed solution comprises one or more of a thickening agent, plasticiser, stabilizer or emulsifier, preferably, wherein the thickening agent and/or plasticiser, and/or stabilizer and/or emulsifier comprises one or more of glycerol, polysorbate 80, modified starch, propylene glycol, preferably polysorbate 80 and/or modified starch and/or propylene glycol.

26.-31. (canceled)
